(19) United States
(12) Patent Application Publication
(10) Pub. No.: US 2008/0269458 A1
(43) Pub. Date: Oct. 30, 2008

(54) POLYIMIDE AND LIQUID CRYSTAL ALIGNMENT FILM THEREOF

(75) Inventors: Yaw-Terng Chern, Taipei (TW); Ya-Ting Wu, Taipei (TW); Jane-Jen Wang, Taipei (TW)

Correspondence Address: VOLPE AND KOENIG, P.C., UNITED PLAZA, SUITE 1600, 30 SOUTH 17TH STREET, PHILADELPHIA, PA, 19103, US

(73) Assignee: National Taiwan University of Science and Technology, Taipei (TW)

(21) Appl. No.: 12/056,385

(22) Filed: Mar. 27, 2008

(30) Foreign Application Priority Data
Apr. 24, 2007 (TW) 096114505

(51) Int. Cl. C08G 73/12; C07C 211/50; C07C 211/52

(52) U.S. Cl. 528/353; 564/300; 564/309

(57) ABSTRACT

The present invention provides a polyimide having the formula (II) formed by reacting a dianhydride with the diamine having the formula (I). The polyimide provided by the present invention serves as a material for preparing the liquid crystal aligning film, wherein the liquid crystal aligning film could achieve the pre-tilt angle of 88-90° by containing only less than 5% of the diamine having the formula (I), which could highly lower down the manufacturing cost.

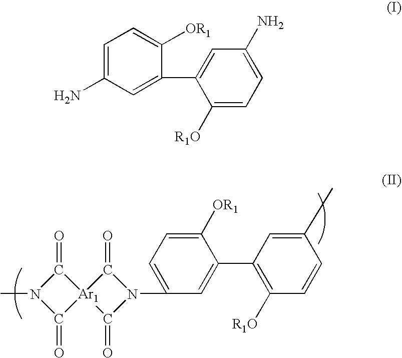

### FIELD OF THE INVENTION

The present invention relates to a polyimide compound, and more particularly to a polyimide compound including alkyl side chains and high reactive diamine, which could apply to the vertical aligning film for soft FPD in the next generation.

### BACKGROUND OF THE INVENTION

Molecular aligning technique is almost applied to all of the liquid crystal elements so that liquid crystals could display the characteristic of specific orientation. Polyimides have good thermal stability, dielectricity and chemical-safety, and thus become the most popular material for the aligning film. For color LCDs, the application of the organic-insoluble polyimide aligning film is limited due to the weak heat-duration of the color filter containing organic dyes. Therefore, there is a need to design the organic soluble polyimides with good aligning stability so that the aligning angle could be adjusted and the voltage could be maintained.

Presently, various kinds of wide viewing angle techniques are provided to achieve a clear watch on the viewing angle ranged from 0 to 160 degree of LCD without color variation among the mentioned angles, wherein the multi-domain vertical alignment (MVA) is widely applied. In order to satisfy the need of MVA LCD, all the aligning film materials that used must have the pre-tilt angle close to 90°, and these vertical aligning film materials could be prepared merely when the polyimides have the specific structure. The current solution is to incorporate a large number of long straight alkyl side chains into the polyimides. However, the surface tension of the above polyimides for the aligning film is lower than 30 dyn/cm, whereby the liquid crystals are hard to be completely wet on the surface of the aligning film, and the displaying quality of LCD is directly influenced thereby. Therefore, it is desirable to solve how to display the vertical aligning pre-tilt angle as well as the incomplete wetting of liquid crystals on the aligning film.

The particular design in the present invention not only solves the problems described above, but also is easy to be implemented. Thus, the invention has the utility for the industry.

### SUMMARY OF THE INVENTION

In order to solve the existing problems of the prior art, the present invention provides a novel and high reactive diamine monomer that is easily polymerized to have high molecular weight. Moreover, the diamine monomer could be reacted to a novel polyimide that displays the vertical aligning effect under a low content of alkyl side chains. Furthermore, the novel diamine monomer developed by the present invention could form an aligning film material with toughness, which could serve as the aligning materials for the soft FPD of the next generation.

In accordance with a first aspect of the present invention, a chemical compound of formula (A) is provided.

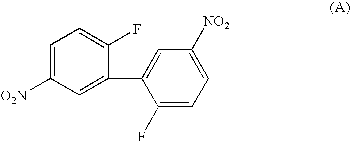

In accordance with a second aspect of the present invention, a diamine compound of formula (I) is provided. The diamine compound is formed by reacting the compound of formula (A) with an alkyl alcohol (R₁OH) under an alkaline condition and then reducing the dinitro-compound:

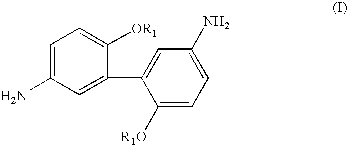

wherein R₁ is a C₂˜C₂₂ alkyl group.

In accordance with a third aspect of the present invention, a polyimide of formula (II) is provided. The polyimide of formula (II) is derived by reacting the diamine compound of formula (I) with a dianhydride containing Ar₁:

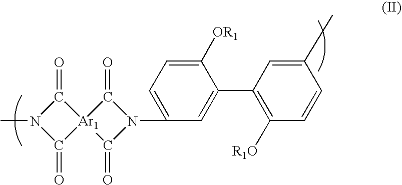

wherein Ar₁ is selected from a group consisting of

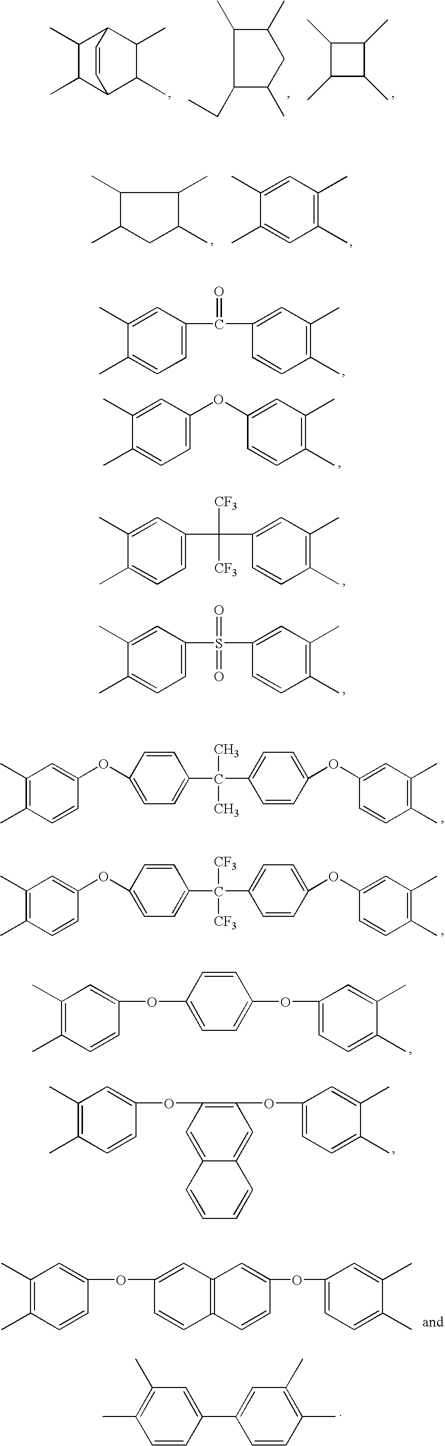

Preferably, the polyimide is dissolvable in a solvent selected from a group consisting of NMP, DMAc,r-butyrolactone, m-cresol, o-chlorophenol, THF and cyclohexanone.

Preferably, the polyimide is dissolvable in NMP.

Preferably, a viscosity of the polyimide is ranged from 0.05 to 3.0 dL/g while the concentration of the polyimide is 0.5 g/dL.

In accordance with a fourth aspect of the present invention, a polyimide copolymer of formula (III) is provided. The polyimide of formula (III) is formed by reacting the diamine compound of formula (II) with a diamine compound containing Ar₂ and a dianhydride containing Ar₁:

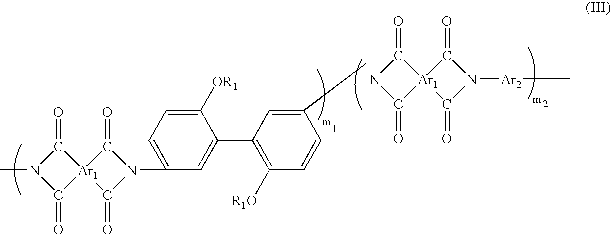

where in m₁ varies from 100 to 1 while m₂ varies correspondingly from 0 to 99; and Ar₁ is selected from a group consisting of

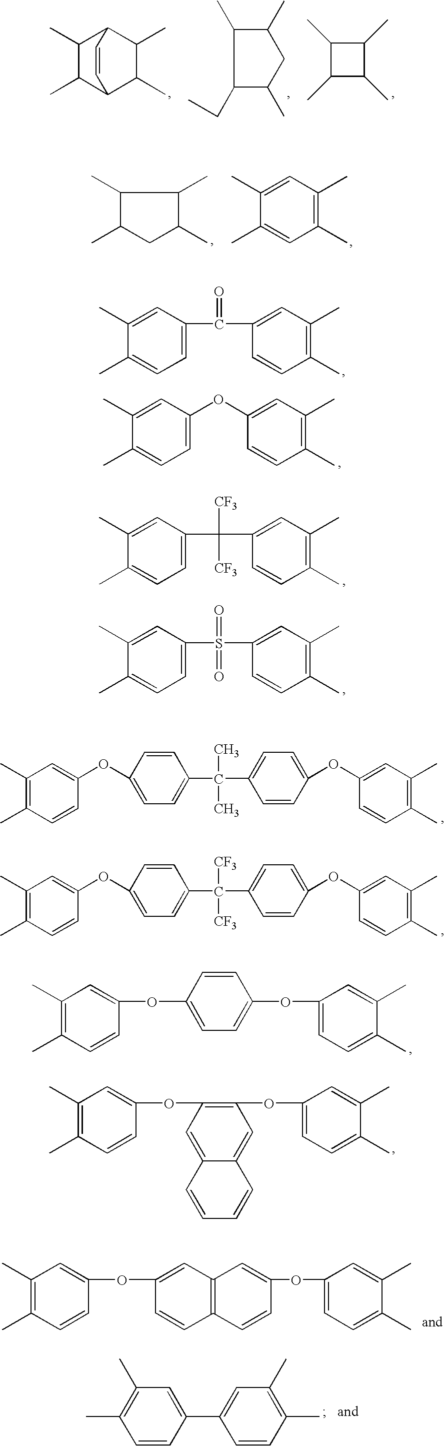

Ar₂ is selected from a group consisting of

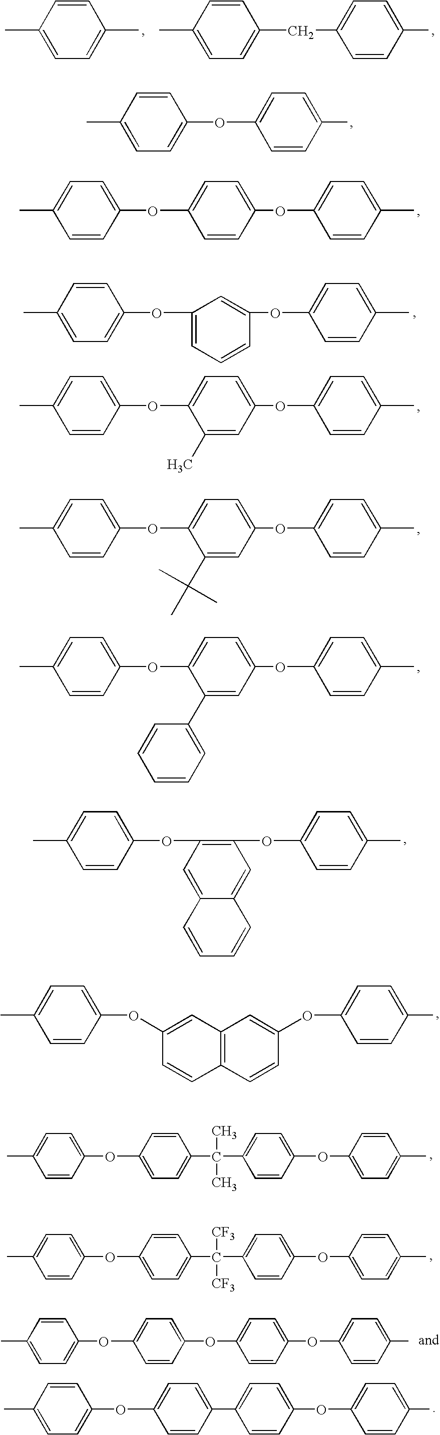

Preferably, the polyimide copolymer is dissolvable in a solvent selected from a group consisting of NMP, DMAc, r-butyrolactone, m-cresol, o-chloroplenol, THF and cyclohexanone.

Preferably, the polyimide copolymer is dissolvable in NMP.

Preferably, a viscosity of the polyimide copolymer is ranged from 0.05 to 3.0 dL/g while the concentration of the polyimide copolymer is 0.5 g/dL.

Preferably, the polyimide copolymer containing the polyimide of formula (II) is 1-100% by weight.

In accordance with a fifth aspect of the present invention, a liquid crystal alignment film comprising the polyimide of formula (II) is provided.

Preferably, the concentration of the polyimide of formula (II) is ranged from 1 to 10%.

Preferably, the concentration of the polyimide of formula (II) is lower than 7%.

In accordance with a sixth aspect of the present invention, a liquid crystal alignment film comprising the polyimide copolymer of formula (III) is provided.

Preferably, the concentration of the polyimide copolymer of formula (III) is ranged from 1 to 10%.

Preferably, the concentration of the polyimide copolymer of formula (III) is lower than 7%.

The above aspects and advantages of the present invention will become more readily apparent to those ordinarily skilled in the art after reviewing the following detailed descriptions.

### DETAILED DESCRIPTION OF THE PREFERRED EMBODIMENT

The present invention will now be described more specifically with reference to the following embodiments. It is to be noted that the following descriptions of preferred embodiments of this invention are presented herein for the purposes of illustration and description only; it is not intended to be exhaustive or to be limited to the precise form disclosed.

A chemical compound of formula (A) is first provided as a starting reactant for the preparation of the liquid crystal alignment film in the present invention.

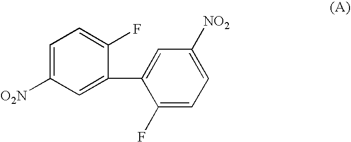

A chemical compound of 3-bromo-4-fluoronitrobezene dissolving in a solvent selected from a group consisting of N,N-dimethylformamide (DMF), dimethyl sulfoxide (DMSO) and N-methyl-2-pyrrolidone (NMP) undergoes an UIImann coupling reaction in the catalysis of copper to obtain the above compound of formula (A).

Based on the mentioned starting reactant, the present invention further provides a diamine compound of formula (I) containing long alkyl side chains.

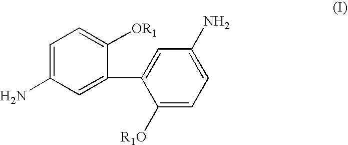

The compound of formula (I) is prepared by (1) performing a nucleophilic reaction between the compound of formula (A) and a long alkyl alcohol (R₁OH) under an alkaline condition to obtain a dinitro-compound with the long alkyl side chains; and (2) reducing the dinitro-compound to the diamine monomer of formula (I). The mentioned alkaline could be selected from one of an inorganic alkaline, such as potassium carbonate, cesium fluoride and etc., and an organic alkaline, such as pyridine. R₁ is a C₂˜C₂₂ alkyl group. These kinds of diamine monomers are especially suitable for the preparation of the polyimide of formula (II) for the vertical alignment film that is formed by reacting the diamine of formula (I) with various kinds of anhydrides.

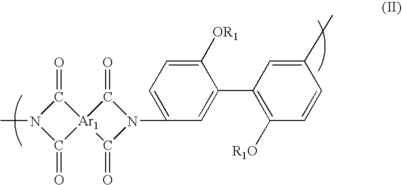

R₁ is a C₂˜C₂₂ alkyl group and Ar₁ is selected from a group consisting of

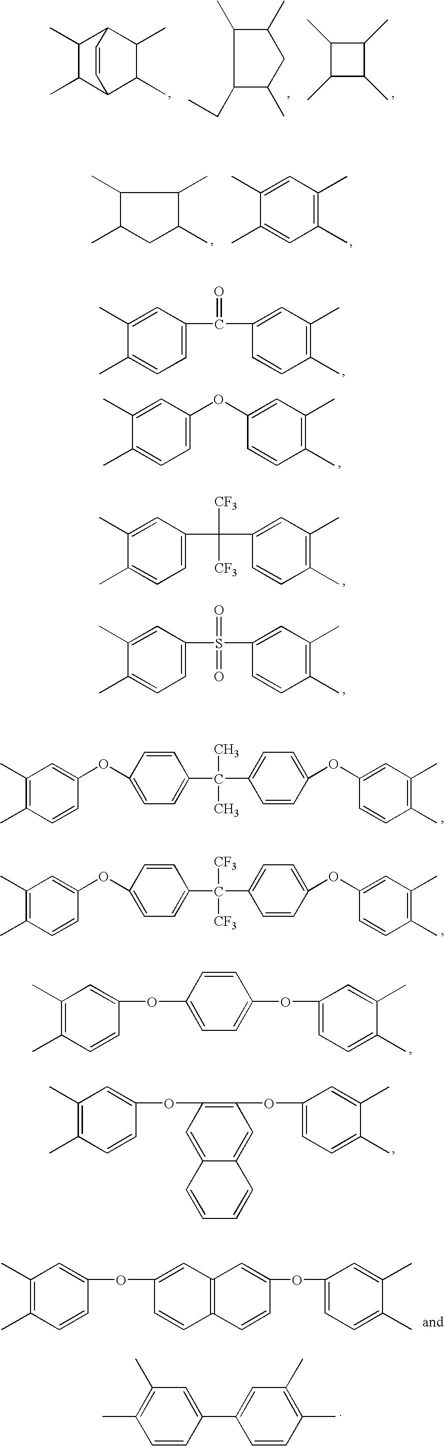

The mentioned polyimides could further be polymerized as a copolymer. Accordingly, the present invention further provides a polyimide copolymer of formula (III) that is formed by reacting the diamine of formula (I) with another diamine compound containing Ar₂ and the dianhydride containing Ar₁ as shown in the following equation:

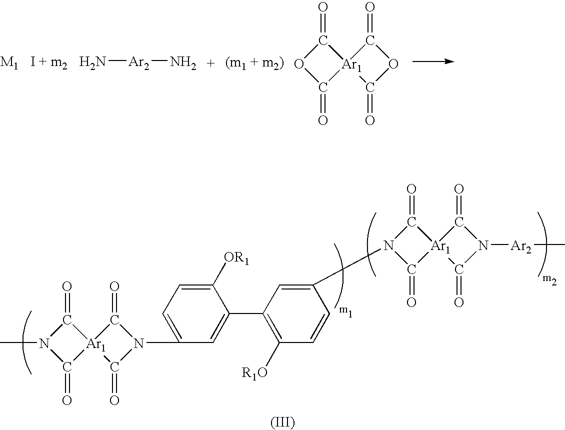

wherein m₁ and m₂ are real numbers, R₁ and Ar₁ are the same as the above and Ar₂ is selected from a group consisting of

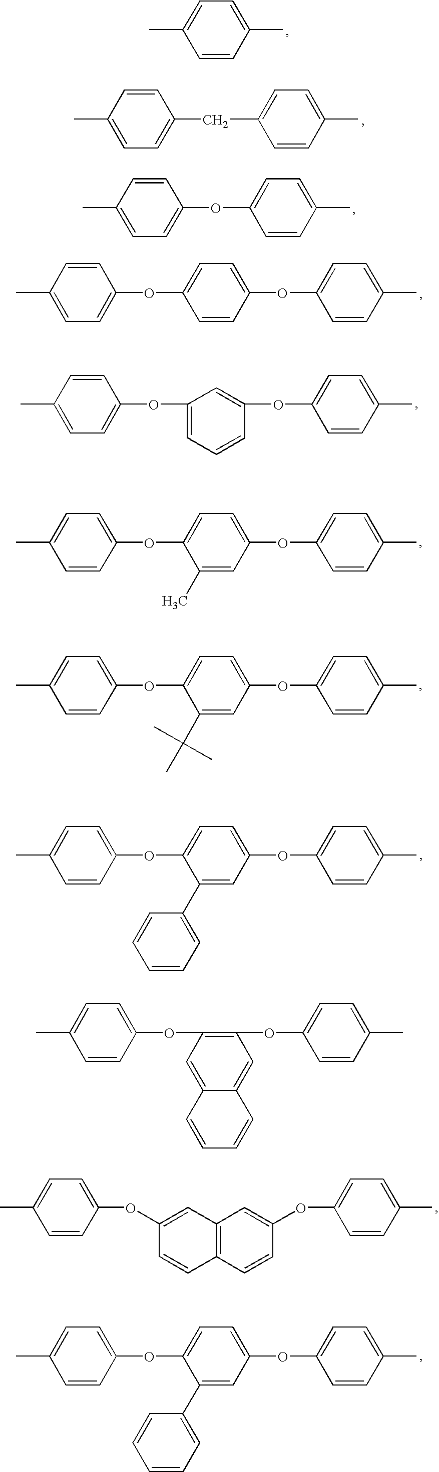

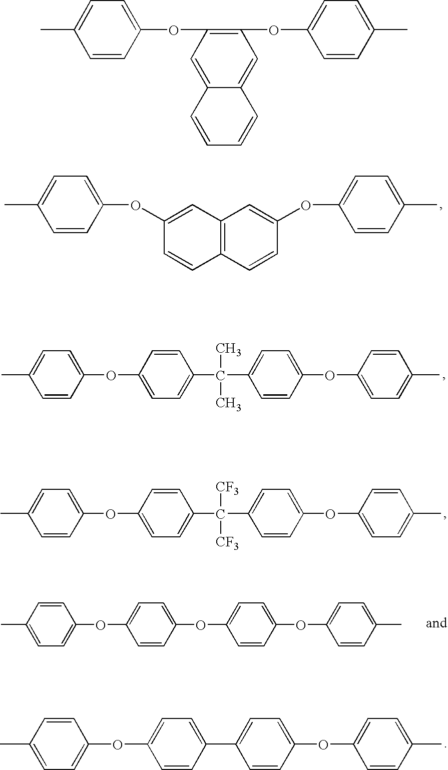

Furthermore, the polyimide copolymer containing the polyimide of formula (II) is 1˜100% by weight.

The preparation of the mentioned polyimide and polyimide copolymer is performed by various kinds of dianhydrides and diamine compounds undergoing a two-step reaction including: (1) an additional polymerization by an open ring to form the polyamic acid, followed by selecting a suitable solvent selecting from a group consisting of NMP, r-butyrolactone, DMAc and m-cresol; (2) a chemical cyclization (such as adding acetic anhydride and pyridine) or heat cyclization. Alternatively, it is available to select a solvent with high boiling point, such as o-xylene or m-cresol, and dissolve the diamine monomer and the dianhydride therein, which produces a co-boiling with water so as to anhydrate the above product and then perform a chemical cyclization. The polyimide and polyimide copolymer could make up a liquid crystal aligning agent with an organic solvent. These liquid crystal aligning agents dissolve in the organic agent that includes aprotic polar solvents and the solvents containing phenol or ether. The concentration of the polyimide and the polyimide copolymer of the present invention for preparing the liquid crystal aligning film depends on the viscosity thereof, wherein the preferred concentration is ranged from 1˜10%. Mounting the liquid crystal aligning film on a working substrate is performed by one of a printing method and a spin coating method, followed by drying the solvent to form the liquid crystal aligning film. However, while the concentration is less than 1%, the thickness of the aligning film is too thin; while the concentration is higher than 10%, the thickness of the aligning film is too thick, both of which fails to make an ideal aligning film.

The liquid crystal display device containing the aligning agent with the polyimide of the present invention is prepared by the following steps:

(1) spin-coating the liquid crystal aligning film with the proper concentration on a glass substrate provided with a transparent electrode(such as ITO glass), followed by removing the solvent through a drying at high temperature ranged from 80 to 250° C.; the thickness of the formed uniform film is usually ranged from 0.001 to 1 μm;

(2) performing a mechanical rubbing for the substrate on the roller with a nylon fiber;

(3) sealing two pieces of the rubbed substrates assembling together in the antiparallel rubbing direction and controlling the gap between the substrate at 25˜50 μm, followed by sealing both sides with a sealing agent; then, injecting the liquid crystal into the hole and being sealed with the sealing agent to prepare the liquid crystal cell. The mentioned sealing agent includes the epoxy resin.

### EXAMPLE 1

### Preparation for 2,2′-difluoro-5,5′-dinitrobiphenyl

100 g (0.454 mol) of 3-bromo-4-fluoronitrobezene was dissolved in a 150 ml solution of DMF and then 60 g of a copper powder was added thereinto, followed by refluxing for 24 hours. The copper powder was filtered out and the resultant solution was cooled to form a precipitate. The precipitate was washed by water where the yield was 65% and the melting point is 199˜201 □. Elemental Anal, Calcd: C, 51.43; H, 2.14, N, 10.00. Found: C, 51.37; H, 2.17; N, 9.96.

### EXAMPLE 2

### Preparation for 2,2′-dihexoxy-5,5′-dinitrobiphenyl

9 g (32.14 mmol) of 2,2′-difluoro-5,5′-dinitrobiphenyl, 8.21g (80.34 mmol) of hexyl alcohol, 4.51 g of potassium hydroxide and 1.33 g (8.01 mmol) of potassium iodide were dissolved in a 60 ml solvent of DMAc for a 24-hour reaction at 120 □, followed by performing a precipitation in water. The precipitate was recrystalized and purified to obtain 2,2′-dihexoxy-5,5′-dinitrobiphenyl. The yield was 55.8% and the melting point is 90˜92 □. Elemental Anal, Calcd: C, 64.86; H, 7.21; N, 6.31. Found: C, 64.84; H, 7.25; N, 6.27.

### EXAMPLE 3

### Preparation for 2,2′-dihexoxy-5,5′-diaminobiphenyl

6 g (13.51 mmol) of 2,2′-dihexoxy-5,5′-dinitrobiphenyl and 0.12 g of Carbon containing 10% Pd (Pd(C)) were added in a 60 ml ethanol solution, followed by adding 30 ml of hydrazine hydrate drop-by-drop at 100 □ thereinto for a two-day reaction. Then, the resultant solution was precipitated in water to further recrystalize and purify the precipitate. The yield was 94.7% and the melting point is 58˜60° C. Elemental Anal, Calcd: C, 75.00; H, 9.38; N, 7.29. Found: C, 74.95; H, 9.42; N, 7.26.

### EXAMPLE 4

### Preparation for 2,2′-didodecoxy-5,5′-dinitrobiphenyl

4 g (14.29 mmol) of 2,2′-difluoro-5,5′-dinitrobiphenyl, 6.65 g (36.84 mmol) of 1-dodecanol and 4.92 g (35.65 mmol) of potassium carbonate were dissolved in a 36 ml DMF solution for a 15-hour refluxation. Then, the resultant solution was precipitated in water to further recrystalize and purify the precipitate. The yield was 60.6% and the melting point is 75˜77° C. Elemental Anal, Calcd: C, 70.59; H, 9.15; N, 4.58. Found: C, 70.55; H, 9.17; N, 4.56.

### EXAMPLE 5

### Preparation for 2,2′-didodecoxy-5,5′-diaminobiphenyl

5.4 g (8.82 mmol) of 2,2′-didodecoxy-5,5′-dinitrobiphenyl and 0.11 g of Carbon containing 10% Pd (Pd(C)) were added in a 54 ml ethanol solution, followed by adding 27 ml of hydrazine hydrate drop-by-drop at 100 □ thereinto for a two-day reaction. Then, the resultant solution was precipitated in water to further recrystalize and purify the precipitate. The yield was 90.3% and the melting point is 58˜60° C. Elemental Anal, Calcd: C, 78.26; H, 10.87; N, 5.07. Found: C, 78.24; H, 10.90; N, 5.03.

### EXAMPLE 6

### Preparation for 2,2′-dioctadecoxy-5,5′-dinitrobiphenyl

The preparation for 2,2′-dioctadecoxy-5,5′-dinitrobiphenyl is similar to the example 4 by replacing 1-dodecanol with 1-octadecanol. The yield was 71.1% and the melting point is 88˜90° C. Elemental Anal, Calcd: C, 73.85; H, 10.26; N, 3.59. Found: C, 73.81; H, 10.30; N, 3.56.

### EXAMPLE 7

### Preparation for 2,2′-dioctadecoxy-5,5′-diaminobiphenyl

The preparation for 2,2′-dioctadecoxy-5,5′-diaminobiphenyl is similar to the example 5 by replacing 2,2′-didodecoxy-5,5′-dinitrobiphenyl with 2,2′-dioctadecoxy-5,5′-dinitrobiphenyl. The yield was 78.9% and the melting point is 82˜84° C. Elemental Anal, Calcd: C, 80.00; H, 11.67; N, 3.89. Found: C, 79.97; H, 11.70; N, 3.85.

### EXAMPLE 8

### Preparation of the Polyimide (II)

0.634 g (1.65 mmol) of 2,2′-dihexyl-5,5′-diaminobiphenyl was dissolved in a 8 ml m-cresol solution, followed by adding 0.490 g (1.65 mmol) of 3,3′,4,4′-biphenyltetracarboxylic dianhydride (BPDA) for a 4-hour reaction at 40° C. Then 8 drops of isoquinoline were added to the resultant solution and the temperature was increased to 210° C. for a 6-hour refluxation. Further, the resultant solution was precipitated in methanol and a Soxhlet extraction was performed with methanol. The polyimide was dried. The inherent viscosity of the polyimide was 0.51 dL/g, as measured at a concentration of 0.5 g/dL in NMP at 30 □. The polymer is dissolvable in one selected from a group consisting of NMP, DMAc, r-butyrolactone, THF and cyclohexanone.

### EXAMPLE 9

### Preparation of the Polyimide (II)

0.909 g (1.65 mmol) of 2,2′-didodecoxy-5,5′-diaminebiphenyl was dissolved in a 8 ml m-cresol solution, followed by adding 0.490 g (1.65 mmol) of 3,3′,4,4′-biphenyltetracarboxylic dianhydride (BPDA) for a 4-hour reaction. Then 8 drops of isoquinoline were added and the temperature was increased to 210° C. for a 6-hour refluxation. Further, the resultant solution was precipitated in methanol and a Soxhlet extraction was performed with methanol. The polyimide was dried. The inherent viscosity of the polyimide was 0.36 dL/g, as measured at a concentration of 0.5 g/dL in NMP at 30 □. The polymer is dissolvable in one selected from a group consisting of NMP, DMAc, r-butylactone, THF and cyclohexanone.

### EXAMPLE 10

### Preparation of the Polyimide (II)

1.188 g (1.65 mmol) of 2,2′- dioctadecoxy-5,5′-diaminebiphenyl was dissolved in a 10 ml m-cresol solution, followed by adding 0.490 g (1.65 mmol) of 3,3′,4,4′-biphenyltetracarboxylic dianhydride (BPDA) for a 4-hour reaction. Then 8 drops of isoquinoline were added and the temperature was increased to 210° C. for a 6-hour refluxation. Further, the resultant solution was precipitated in methanol and a Soxhlet extraction was performed with methanol. The extract was dried. The inherent viscosity of the polyimide was 0.61 dL/g, as measured at a concentration of 0.5 g/dL in NMP at 30 □. The polymer is dissolvable in one selected from a group consisting of NMP, DMAc, r-butyrolactone, THF and cyclohexanone.

### EXAMPLE 11

### Embodiments of the Preparation for Polyimide Copolymer

The illustration for the abbreviations, x C_{y}O_{z}A, is provided as below:

x: the carbon number of the diamine monomer of the alkyl side chains (x=18, 12, 6)

C_{y}: the molar percentage of the diamine monomer of the alkyl side chains by the total diamine monomer

O_{z}: the molar percentage of 4,4′-Oxydianiline (ODA) by the total diamine monomer

A: the polymerized dianhydride (the abbreviations are listed as below)

C represents 2,3,5-tricarboxycyclopentylacetic acid dianhydride (CPDA); P represents pyromellitic dianhydride (PMDA); B represents 3,3′,4,4′-Biphenyltetracarboxylic dianhydride (BPDA); O represents 4,4′- Oxydiphthalic anhydride (ODPA); F represents 4,4′-(Hexafluoro-isopropylidene)diphthalic anhydride (6FDA) and BT represents 3,3′,4,4′-Benzophenonetetracarboxylic dianhydride (BTDA).

The polymer of 18C₂₀O₈₀C was taken as an exemplary example, which represents the polymer comprises the C₁₈ diamine monomer of the alkyl side chains polymerized with ODA and CPDA and the molar ratio of the C₁₈ diamine monomer to ODA is 20:80.

### EXAMPLE 12

### Chemical Cyclization of 12C₃₀O₇₀C

0.4690 g (0.8496 mmol) of 2,2′- didodecoxy-5,5′-diaminebiphenyl and 0.3965 g (1.983 mmol) of ODA were dissolved in a 8 ml m-cresol solution, followed by adding 0.6471 g (2.889 mmol) of CPDA for a 4-hour reaction at 40° C. Then 8 drops of isoquinoline were added and the temperature was increased to 180° C. for a 5-hour refluxation. Further, the resultant solution was precipitated in methanol and a Soxhlet extraction was performed with methanol. The polyimide was dried. The inherent viscosity of the polyimide was 0.39 dL/g, as measured at a concentration of 0.5 g/dL in NMP at 30 □. The polymer is dissolvable in one selected from a group consisting of NMP, DMAc, r-butyrolactone, THF and cyclohexanone.

### EXAMPLE 13

### Thermal Cyclization of 18 C₃₀O₇₀P

0.3763 g (0.5226 mmol) of 2,2′-dioctadecoxy-5,5′-diaminebiphenyl and 0.2439 g (1.219 mmol) of ODA were dissolved in a 10 ml m-cresol solution, followed by adding 0.3836 g (1.759 mmol) of PMDA for a 6-hour reaction at room temperature. The inherent viscosity of the polyamic acid was 1.03 dL/g, as measured at a concentration of 0.5 g/dL in NMP at 30 □. Then the polymer was washed with water and dried.

### EXAMPLE 14

The inherent viscosity and the solubility of the mentioned polyimide and the polyimide copolymer were respectively measured based on the above preparations so as to serve as a determination factor whether it is suitable for the material of vertical aligning films.

Please refer to Table 1, which shows the inherent viscosity of different embodiments of the polyimide of the present invention. Embodiments 1-1˜1-5 show the measurements of the C₁₈ diamine monomer of formula (I) respectively polymerized with CPDA, PMDA, BPDA, ODPA and 6FDA. Embodiments 2-1˜2-5 show the measurements of the C₁₂ diamine monomer of formula (I) respectively polymerized with CPDA, PMDA, BPDA, ODPA and 6FDA. Similarly, embodiments 3-1˜3-5 show the measurements of the C₆ diamine monomer of formula (I) respectively polymerized with CPDA, PMDA, BPDA, ODPA and 6FDA. The inherent viscosities of polyimides were measured at a concentration of 0.5 g/dL in NMP at 30 □.

Please refer to Table 2, which shows the inherent viscosities of the polyimide copolymer of different embodiments of the present invention. Embodiments 1′-1˜1′-5, 2′-1˜2′-5 and 3′-1˜3′-5 show the measurements of the polyimide copolymers with the respective C₁₈, C₁₂ and C₆ diamine monomers of formula (I) polymerized with ODA and BPDA, wherein ODAs are 95%, 90%, 80%, 70% and 60% by molar weight of the total diamine monomers. Embodiments 4′-1˜4′-5, 5′-1˜5′-5 and 6′-1˜6′-5 show the measurements of the polyimide copolymers with the respective C₁₈, C₁₂ and C₆ diamine monomers of formula (I) polymerized with ODA and PMDA, wherein ODAs are 95%, 90%, 80%, 70% and 60% by molar weight of the total diamine monomers. Embodiments 7′-1˜7′-5, 8′-1˜8′-5 and 9′-1˜9′-5, show the measurements of the polyimide copolymers with the respective C₁₈, C₁₂ and C₆ diamine monomers of formula (I) polymerized with ODA and CPDA, wherein ODAs are 95%, 90%, 80%, 70% and 60% by molar weight of the total diamine monomers. Embodiments 10′-3˜10′-5, 11′-1˜11′-5 and 12′-1˜12′-5 show the measurements of the polyimide copolymers with the respective C₁₈, C₁₂ and C₆ diamine monomers of formula (I) polymerized with ODA and OPDA, wherein ODAs are 80%, 70% and 60% by molar weight of the total diamine monomers. Embodiments 13′-3˜13′-5 and 14′-1˜14′-5 show the measurements of the polyimide copolymers with the respective C₁₈ and C₁₂ diamine monomers of formula (I) polymerized with ODA and 6FDA, wherein ODAs are 80%, 70% and 60% by molar weight of the total diamine monomers. These inherent viscosities of polyimide copolymers were measured at a concentration of 0.5 g/dL in NMP at 30 □.

It is known from the measurements as illustrated in Tables 1 and 2 that the diamine monomers of the alkyl side chains provided by the present invention are highly reactive and easily polymerized to form the polymer with high inherent viscosity.

### EXAMPLE 15

Please refer to Table 3, which shows the quantitative solubility of the present polyimide dissolved in the respective NMP, DMAc, m-cresol, o-chlorophenol, acetone, THF, chloroform and cyclohexanone. The measurements were performed by adding 0.01 g of the present polyimide to 1 ml of the mentioned organic solvents. The symbol, “++”, represents that it is soluble at the room temperature; the symbol, “+”, represents that it is soluble when the temperature is raised to 60° C.; the symbol, “±”, represents that it is partially soluble when the temperature was raised to 60° C.; and the symbol, “−”, represents that it is not soluble even the temperature was raised.

Please refer to Table 4, which shows the quantitative solubility of the present polyimide copolymer dissolved in the respective NMP, DMAc, m-cresol, o-chlorophenol, acetone, THF, chloroform and cyclohexanone. The measurements were performed in the way the same as Table 3. It is known from the measurements of Tables 3 and 4 that the present polyimide and the polyimide copolymer have the better solubility since most they were soluble in the tested solvent at the room temperature.

After measuring the respective inherent viscosity and the solubility of the present polyimide and the polyimide copolymer, those with suitable chemical properties for the liquid crystal aligning film were further selected and their pre-tilting angles were measured.

### EXAMPLE 16

### Measurement for Thermal Property

Glass transition temperature (Tg) of the present polyimide and the polyimide copolymer were measured respectively by a differential scanning calorimeter (DSC) and a dynamic mechanical analyzer (DMA). The thermal stability of the present polyimide and the polyimide copolymer were measured by a thermogravimetric analyzer (TGA). It is known from the Table 5 shows that the Tg of the polyimide and the polyimide copolymer of the present invention are above 200° C. except for the embodiment 1′-5. That is, the polyimide and the polyimide copolymer of the present invention have high Tg. In addition, the temperature at which 10% weight loss recorded by TGA at a heating rate of 10° C./min is higher than 400° C. That is, the polyimide and the polyimide copolymer of the present invention also have good thermal stability no matter under the nitrogen gas or the atmosphere based on the result measured by TGA. Based on the mentioned results, the polyimide and the polyimide copolymer of the present invention have good thermal properties. For applying to the material of the aligning film for TFT displays, the good thermal properties of the present polymer is capable of maintaining the thermal stability and the aligning reliability while the temperature of these TFT displays is elevated due to the long-term usage.

### EXAMPLE 17

### Measurement for the Pre-Tilting Angle

The organic-soluble polyimide was dissolved in a solvent including NMP and 2-ethoxyethanol (5:1) and the concentration thereof was designed to 6 w/v %. The mentioned solution was spin-coated on the ITO glass at 2500 rpm and heat-treated at 90° C. for 10 minutes and 180° C. for one hour to obtain the uniform film whose thickness was approximately 1000 Å. Moreover, regarding the organic-insoluble and soluble polyimide of the present invention, the similar preparation was adopted. The polyamic acid was dissolved in a solvent including NMP and 2-ethoxyethanol (5:1) and the concentration thereof was designed to 6 w/v %. The mentioned solution was spin-coated on the ITO glass at 2500 rpm and heat-treated at 90° C. for 10 minutes, 180° C. for one hour and then 250° C. for one hour to form a uniform polyimide coating film,

The coating film was rubbed with a cloth. Then, two pieces of the rubbed substrates were assembled together in the antiparallel rubbing direction, followed by controlling the gap between the substrates at 50 μm. Then, the liquid crystal of ZLI-2293 (produced by Merck) was injected into the space to obtain the liquid-crystal cell.

The pre-tilting angle was measured by an Autronic TBA107 instrument, which was calculated by detecting the chiral center of the optical anisotropy of the liquid crystal molecules by means of the liquid-crystal cell. Further, the liquid-crystal cell was baked at 60° C. for 72 hours. The pre-tilting angle was re-measured so that a stable aligning effect of the film at high temperature could be determined. All the measurements are respectively listed in Table 6 to Table 9.

According to the results as shown in Table 6 to Table 9, it was found that the polyimide copolymer containing C₁₈ side chains can display the vertical alignment no matter polymerized with any kinds of anhydride merely at 5% by weight and has good stability; the polyimide copolymer containing C₁₂ side chains only can display the vertical alignment when the concentration of the long chain monomer is raised, wherein the polyimide copolymer polymerized with CPDA displays the vertical alignment when the concentration of the long chain monomer is lower (10%); the polyimide copolymer containing C₆ side chains fails to display the vertical alignment under no rubbing at the tested concentration of the long chain monomer. Furthermore, it was also found that the polyimide copolymer polymerized with CPDA displays the vertical alignment when the concentration of the long chain monomer is low (10%), but those polymerized with BPDA does when the concentration is 20% and those polymerized with PMDA does when the concentration is 30%.

### EXAMPLE 18

### Reliability Testing for Pre-Tiliting Angle

Please refer to Table 10, which shows a reliability testing for the measurements recorded in Tables 6-9. The result of Table 10 indicates that the aligning films were prepared by the polyimide copolymers of the present embodiments mounted on the liquid-crystal cells that used for long-term still display the stable aligning effect, except for those polymerized with 6FDA that have a declining phenomena under a long-term baking.

In conclusion, the polyimide material for the liquid crystal aligning film can solve the issue of wide-viewing angles of large size TFT LCD and merely 5% of the diamine monomer included therein can display the pre-tilting angle of 88˜90° without the mechanical rubbing. Therefore, the material for the liquid crystal aligning film provided by the present invention can highly reduce the manufacturing cost of the LCD.

While the invention has been described in terms of what is presently considered to be the most practical and preferred embodiments, it is to be understood that the invention needs not be limited to the disclosed embodiments. On the contrary, it is intended to cover various modifications and similar arrangements included within the spirit and scope of the appended claims which are to be accorded with the broadest interpretation so as to encompass all such modifications and similar structures.

TABLE 1
The Inherent Viscosities of Polyimides

| Embodiments | Polymer | ηᵢₙₕ(^{dL}/_{g})^{a} |
|---|---|---|
| 1-1 | 18C₁₀₀O₀C | 0.19 |
| 1-2 | 18C₁₀₀O₀B | 0.61 |
| 1-3 | 18C₁₀₀O₀P | 0.88 |
| 1-4 | 18C₁₀₀O₀O | 0.63 |
| 1-5 | 18C₁₀₀O₀F | 1.22 |
| 2-1 | 12C₁₀₀O₀C | 0.35 |
| 2-2 | 12C₁₀₀O₀B | 0.36 |
| 2-3 | 12C₁₀₀O₀P | 0.67 |
| 2-4 | 12C₁₀₀O₀O | 0.42 |
| 2-5 | 12C₁₀₀O₀F | 0.87 |
| 3-1 | 6C₁₀₀O₀C | 0.28 |
| 3-2 | 6C₁₀₀O₀B | 0.51 |
| 3-3 | 6C₁₀₀O₀P | 1.09 |
| 3-4 | 6C₁₀₀O₀O | 0.69 |
| 3-5 | 6C₁₀₀O₀F | 0.81 |

^{a}Measured in NMP on 0.5 g/_{dL} at 30° C.

TABLE 2
The Inherent Viscosities of Polyimide Copolymer

| | | Inherent |
|---|---|---|
| | | Viscosities^{a} |
| Embodiments | Polyimide Copolymer | [ηᵢₙₕ(^{dL}/_{g})] |
| 1′-1 | 18C₅O₉₅B | 1.12 |
| 1′-2 | 18C₁₀O₉₀B | 1.16 |
| 1′-3 | 18C₂₀O₈₀B | 0.91 |
| 1′-4 | 18C₃₀O₇₀B | 0.68 |
| 1′-5 | 18C₄₀O₆₀B | 0.78 |
| 2′-1 | 12C₅O₉₅B | 1.15 |
| 2′-2 | 12C₁₀O₉₀B | 1.33 |
| 2′-3 | 12C₂₀O₈₀B | 0.80 |
| 2′-4 | 12C₃₀O₇₀B | 0.89 |
| 2′-5 | 12C₄₀O₆₀B | 0.77 |
| 3′-1 | 6C₅O₉₅B | 1.12 |
| 3′-2 | 6C₁₀O₉₀B | 1.16 |
| 3′-3 | 6C₂₀O₈₀B | 0.50 |
| 3′-4 | 6C₃₀O₇₀B | 0.67 |
| 3′-5 | 6C₄₀O₆₀B | 0.78 |
| 4′-1 | 18C₅O₉₅P | 0.75 |
| 4′-2 | 18C₁₀O₉₀P | 0.64 |
| 4′-3 | 18C₂₀O₈₀P | 0.96 |
| 4′-4 | 18C₃₀O₇₀P | 1.03 |
| 4′-5 | 18C₄₀O₆₀P | 0.61 |
| 5′-1 | 12C₅O₉₅P | 0.70 |
| 5′-2 | 12C₁₀O₉₀P | 0.76 |
| 5′-3 | 12C₂₀O₈₀P | 1.40 |
| 5′-4 | 12C₃₀O₇₀P | 1.47 |
| 5′-5 | 12C₄₀O₆₀P | 1.53 |
| 6′-1 | 6C₅O₉₅P | 0.35 |
| 6′-2 | 6C₁₀O₉₀P | 0.41 |
| 6′-3 | 6C₂₀O₈₀P | 0.77 |
| 6′-4 | 6C₃₀O₇₀P | 0.80 |
| 6′-5 | 6C₄₀O₆₀P | 0.88 |
| 7′-1 | 18C₅O₉₅C | 0.32 |
| 7′-2 | 18C₁₀O₉₀C | 0.30 |
| 7′-3 | 18C₂₀O₈₀C | 0.48 |
| 7′-4 | 18C₃₀O₇₀C | 0.26 |
| 7′-5 | 18C₄₀O₆₀C | 0.21 |
| 8′-1 | 12C₅O₉₅C | 0.44 |
| 8′-2 | 12C₁₀O₉₀C | 0.48 |
| 8′-3 | 12C₂₀O₈₀C | 0.61 |
| 8′-4 | 12C₃₀O₇₀C | 0.39 |
| 8′-5 | 12C₄₀O₆₀C | 0.32 |
| 9′-1 | 6C₅O₉₅C | 0.35 |
| 9′-2 | 6C₁₀O₉₀C | 0.41 |
| 9′-3 | 6C₂₀O₈₀C | 0.45 |
| 9′-4 | 6C₃₀O₇₀C | 0.35 |
| 9′-5 | 6C₄₀O₆₀C | 0.30 |
| 10′-3 | 18C₂₀O₈₀O | 0.80 |
| 10′-4 | 18C₃₀O₇₀O | 0.46 |
| 10′-5 | 18C₄₀O₆₀O | 0.67 |
| 11′-3 | 12C₂₀O₈₀O | 0.54 |
| 11′-4 | 12C₃₀O₇₀O | 0.67 |
| 11′-5 | 12C₄₀O₆₀O | 0.87 |
| 12′-3 | 6C₂₀O₈₀O | 0.96 |
| 12′-4 | 6C₃₀O₇₀O | 0.76 |
| 12′-5 | 6C₄₀O₆₀O | 0.62 |
| 13′-3 | 18C₂₀O₈₀F | 0.74 |
| 13′-4 | 18C₃₀O₇₀F | 0.72 |
| 13′-5 | 18C₄₀O₆₀F | 0.82 |
| 14′-3 | 12C₂₀O₈₀F | 0.53 |
| 14′-4 | 12C₃₀O₇₀F | 0.42 |
| 14′-5 | 12C₄₀O₆₀F | 0.48 |

^{a}Measured in NMP on 0.5 g/_{dL} at 30° C.

TABLE 3
Solubility of Polyimides

| Embodiments | Polyimide | A | B | C | D | E | F | G | H |
|---|---|---|---|---|---|---|---|---|---|
| 1-1 | 18C₁₀₀O₀C | ++ | ++ | ++ | ++ | − | ++ | ++ | ++ |
| 1-2 | 18C₁₀₀O₀B | + | + | ++ | ++ | − | ++ | ++ | ++ |
| 1-3 | 18C₁₀₀O₀P | + | + | + | ++ | − | ++ | ++ | ++ |
| 1-4 | 18C₁₀₀O₀O | + | + | ++ | ++ | − | ++ | ++ | ++ |
| 1-5 | 18C₁₀₀O₀F | + | + | ++ | ++ | − | ++ | ++ | ++ |
| 2-1 | 12C₁₀₀O₀C | ++ | ++ | ++ | ++ | − | ++ | ++ | ++ |
| 2-2 | 12C₁₀₀O₀B | ++ | ++ | ++ | ++ | − | ++ | ++ | ++ |
| 2-3 | 12C₁₀₀O₀P | + | + | + | ± | − | ++ | ++ | + |
| 2-4 | 12C₁₀₀O₀O | + | + | ++ | ++ | − | ++ | ++ | ++ |
| 2-5 | 12C₁₀₀O₀F | ++ | ++ | ++ | ++ | − | ++ | ++ | ++ |
| 3-1 | 6C₁₀₀O₀C | ++ | ++ | ++ | ++ | − | ++ | ++ | ++ |
| 3-2 | 6C₁₀₀O₀B | ++ | ++ | ++ | ++ | − | + | ++ | ++ |
| 3-3 | 6C₁₀₀O₀P | ++ | ++ | ++ | ++ | − | ++ | ++ | ++ |
| 3-4 | 6C₁₀₀O₀O | ++ | ++ | ++ | ++ | − | ++ | ++ | ++ |
| 3-5 | 6C₁₀₀O₀F | ++ | ++ | ++ | ++ | ± | ++ | ++ | ++ |

A: NMP;
B: DMAc;
C: m-cresol;
D: o-Chlorophenol;
E: acetone;
F: THF;
G: chloroform;
H: cyclohexanone
Qualitative solubility was determined using 0.01 g of polymer in 1 mL of solvent.
++; soluble at room temperature;
+: soluble on heating at 60° C.;
±: partially soluble on heating at 60° C.;
−: insoluble in hot solvent.

TABLE 4
Solubility of Polyimide Copolymer

| | Polyimide | | | | | | | | |
|---|---|---|---|---|---|---|---|---|---|
| Embodiments | Copolymer | A | B | C | D | E | F | G | H |
| 7′-1 | 18C₅O₉₅C | ++ | ++ | + | ++ | − | ++ | − | ++ |
| 7′-2 | 18C₁₀O₉₀C | ++ | ++ | + | ++ | − | ++ | − | ++ |
| 7′-3 | 18C₂₀O₈₀C | ++ | ++ | + | ++ | − | ++ | − | ++ |
| 7′-4 | 18C₃₀O₇₀C | ++ | ++ | ++ | ++ | − | ++ | − | ++ |
| 7′-5 | 18C₄₀O₆₀C | ++ | ++ | ++ | ++ | − | ++ | − | ++ |
| 10′-3 | 18C₂₀O₈₀O | ++ | ++ | ++ | + | − | ++ | ++ | ++ |
| 10′-4 | 18C₃₀O₇₀O | ++ | ++ | ++ | + | − | ++ | ++ | ++ |
| 10′-5 | 18C₄₀O₆₀O | ++ | + | ++ | + | − | ++ | ++ | ++ |
| 13′-3 | 18C₂₀O₈₀F | ++ | ++ | ++ | ++ | + | ++ | ++ | ++ |
| 13′-4 | 18C₃₀O₇₀F | ++ | ++ | ++ | ++ | ++ | ++ | ++ | ++ |
| 13′-5 | 18C₄₀O₆₀F | ++ | ++ | ++ | ++ | ++ | ++ | ++ | ++ |
| 8′-1 | 12C₅O₉₅C | ++ | ++ | + | + | − | ++ | − | ++ |
| 8′-2 | 12C₁₀O₉₀C | ++ | ++ | + | + | − | ++ | − | ++ |
| 8′-3 | 12C₂₀O₈₀C | ++ | ++ | + | + | − | ++ | − | ++ |
| 8′-4 | 12C₃₀O₇₀C | ++ | ++ | + | + | − | ++ | − | ++ |
| 8′-5 | 12C₄₀O₆₀C | ++ | ++ | + | + | − | ++ | − | ++ |
| 11′-3 | 12C₂₀O₈₀O | ++ | ++ | ++ | + | − | ++ | ++ | ++ |
| 11′-4 | 12C₃₀O₇₀O | ++ | ++ | + | + | − | ++ | ++ | ++ |
| 11′-5 | 12C₄₀O₆₀O | ++ | ++ | + | + | − | ++ | ++ | ++ |
| 14′-3 | 12C₂₀O₈₀F | ++ | ++ | ++ | ++ | ± | ++ | ++ | ++ |
| 14′-4 | 12C₃₀O₇₀F | ++ | ++ | ++ | ++ | ++ | ++ | ++ | ++ |
| 14′-5 | 12C₄₀O₆₀F | ++ | ++ | ++ | ++ | ++ | ++ | ++ | ++ |
| 9′-1 | 6C₅O₉₅C | ++ | + | + | ± | − | ± | − | ++ |
| 9′-2 | 6C₁₀O₉₀C | ++ | + | + | + | − | + | − | ++ |
| 9′-3 | 6C₂₀O₈₀C | ++ | + | + | + | − | ++ | − | ++ |
| 9′-4 | 6C₃₀O₇₀C | ++ | + | + | + | − | ++ | − | ++ |
| 9′-5 | 6C₄₀O₆₀C | ++ | ++ | + | + | − | ++ | − | ++ |
| 12′-3 | 6C₂₀O₈₀O | ++ | + | ++ | ± | − | ± | ++ | ++ |
| 12′-4 | 6C₃₀O₇₀O | ++ | ++ | ++ | + | − | + | ++ | ++ |

A: NMP;
B: DMAc;
C: m-cresol;
D: o-Chlorophenol;
E: acetone;
F: THF;
G: chloroform;
H: cyclohexanone
Qualitative solubility was determined using 0.01 g of polymer in 1 mL of solvent.
++; soluble at room temperature;
+: soluble on heating at 60° C.;
±: partially soluble on heating at 60° C.;
−: insoluble in hot solvent.

TABLE 5
Thermal Properties of Polymers

| | | | | | | char |
|---|---|---|---|---|---|---|
| | | Tg (° C.) | | Td (° C.)^{c} | | yield^{d} |
| Embodiments | Polymer | DSC^{a} | DMA^{b} | In Air | In N₂ | (%) |
| 1-1 | 18C₁₀₀O₀C | 206 | — | 362 | 360 | 23 |
| 2-1 | 12C₁₀₀O₀C | 221 | — | 406 | 404 | 26 |
| 3-1 | 6C₁₀₀O₀C | 245 | — | 402 | 398 | 38 |
| 1′-1 | 18C₅O₉₅B | 256 | 268 | 563 | 551 | 60 |
| 1′-4 | 18C₃₀O₇₀B | 202 | 225 | 443 | 435 | 48 |
| 1′-5 | 18C₄₀O₆₀B | 191 | 216 | 434 | 429 | 45 |
| 2′-1 | 12C₅O₉₅B | 258 | 275 | 574 | 558 | 59 |
| 2′-3 | 12C₂₀O₈₀B | 225 | 241 | 461 | 460 | 56 |
| 2′-4 | 12C₃₀O₇₀B | 206 | 229 | 447 | 446 | 53 |
| 2′-5 | 12C₄₀O₆₀B | 201 | 220 | 439 | 439 | 51 |
| 3′-3 | 6C₂₀O₈₀B | 270 | 270 | 536 | 526 | 59 |
| 3′-4 | 6C₃₀O₇₀B | 251 | 263 | 470 | 468 | 56 |
| 3′-5 | 6C₄₀O₆₀B | 234 | 252 | 443 | 441 | 53 |

^{a}Temperature at which the middle of change of the heat capacity occurred from the second DSC heating scan at a heating rate of 20° C./min.
^{b}Temperature at which the peak maximum of tanδ occurred as recorded by DMA at a heating of 5° C./min.
^{c}Temperature at which 10% weight loss recorded by thermogravimetry at a heating rate of 10° C./min.
^{d}Residual weight % at 700° C. in nitrogen.

TABLE 6
Pre-tilting angles of the polyimide copolymer polymerized with CPDA

| | | | | Aligning |
|---|---|---|---|---|
| Embodiments | Polymer | Unrubbed | Rubbed | characteristics |
| 7′-1 | 18C₅O₉₅C | 88.6° | 89.7° | Excellent |
| 7′-2 | 18C₁₀O₉₀C | 88.3° | 88.5° | Excellent |
| 7′-3 | 18C₂₀O₈₀C | 88.5° | 88.8° | Excellent |
| 7′-4 | 18C₃₀O₇₀C | 88.6° | 89.5° | Excellent |
| 7′-5 | 18C₄₀O₆₀C | 88.5° | 89.6° | Excellent |
| 8′-1 | 12C₅O₉₅C | 0.80° | 10.4° | Excellent |
| 8′-2 | 12C₁₀O₉₀C | 88.5° | 89.8° | Excellent |
| 8′-3 | 12C₂₀O₈₀C | 88.0° | 89.4° | Excellent |
| 8′-4 | 12C₃₀O₇₀C | 88.8° | 89.2° | Excellent |
| 8′-5 | 12C₄₀O₆₀C | 88.4° | 89.8° | Excellent |
| 9′-1 | 6C₅O₉₅C | 0.6° | 7.2° | Excellent |
| 9′-2 | 6C₁₀O₉₀C | 0.9° | 87.8° | Excellent |
| 9′-3 | 6C₂₀O₈₀C | 11.2° | 88.3° | Excellent |
| 9′-4 | 6C₃₀O₇₀C | 11.0° | 88.7° | Excellent |
| 9′-5 | 6C₄₀O₆₀C | 12.6° | 89.3° | Excellent |

TABLE 7
Pre-tilting angles of the polyimide copolymer polymerized with BPDA

| | | | | Aligning |
|---|---|---|---|---|
| Embodiments | Polymer | Unrubbed | Rubbed | characteristics |
| 1′-1 | 18C₅O₉₅B | 88.5° | 89.9° | Excellent |
| 1′-2 | 18C₁₀O₉₀B | 88.1° | 89.6° | Excellent |
| 1′-3 | 18C₂₀O₈₀B | 87.7° | 87.7° | Excellent |
| 1′-4 | 18C₃₀O₇₀B | 88.0° | 88.1° | Excellent |
| 1′-5 | 18C₄₀O₆₀B | 88.4° | 88.9° | Excellent |
| 2′-1 | 12C₅O₉₅B | 0.5° | 6.4° | Excellent |
| 2′-2 | 12C₁₀O₉₀B | 5.9° | 16.2° | Excellent |
| 2′-3 | 12C₂₀O₈₀B | 87.6° | 88.5° | Excellent |
| 2′-4 | 12C₃₀O₇₀B | 89.1° | 88.9° | Excellent |
| 2′-5 | 12C₄₀O₆₀B | 88.3° | 88.5° | Excellent |
| 3′-1 | 6C₅O₉₅B | 0.2° | 4.2° | Excellent |
| 3′-2 | 6C₁₀O₉₀B | 1.7° | 8.7° | Excellent |
| 3′-3 | 6C₂₀O₈₀B | 4.6° | 88.7° | Excellent |
| 3′-4 | 6C₃₀O₇₀B | 6.9° | 89.6° | Excellent |
| 3′-5 | 6C₄₀O₆₀B | 6.1° | 89.6° | Excellent |

TABLE 8
Pre-tilting angles of the polyimide copolymer polymerized with PMDA

| | | | | Aligning |
|---|---|---|---|---|
| Embodiments | Polymer | Unrubbed | Rubbed | characteristics |
| 4′-1 | 18C₅O₉₅P | 87.3° | 89.0° | Excellent |
| 4′-2 | 18C₁₀O₉₀P | 88.5° | 89.3° | Excellent |
| 4′-3 | 18C₂₀O₈₀P | 88.5° | 89.5° | Excellent |
| 4′-4 | 18C₃₀O₇₀P | 89.1° | 89.2° | Excellent |
| 4′-5 | 18C₄₀O₆₀P | 88.9° | 89.6° | Excellent |
| 5′-1 | 12C₅O₉₅P | 0.9° | 1.3° | Excellent |
| 5′-2 | 12C₁₀O₉₀P | 1.1° | 6.8° | Excellent |
| 5′-3 | 12C₂₀O₈₀P | 88.3° | 89.3° | Excellent |
| 5′-4 | 12C₃₀O₇₀P | 88.0° | 89.5° | Excellent |
| 5′-5 | 12C₄₀O₆₀P | 88.3° | 89.6° | Excellent |
| 6′-1 | 6C₅O₉₅P | 0.3° | 0.5° | Excellent |
| 6′-2 | 6C₁₀O₉₀P | 0.9° | 1.0° | Excellent |
| 6′-3 | 6C₂₀O₈₀P | 0.9° | 1.2° | Excellent |
| 6′-4 | 6C₃₀O₇₀P | 8.4° | 89.1° | Excellent |
| 6′-5 | 6C₄₀O₆₀P | 8.3° | 89.2° | Excellent |

TABLE 9
Pre-tilting angles of the polyimide copolymer polymerized with
ODPA, BTDA and 6FDA

| | | | | Aligning |
|---|---|---|---|---|
| Embodiments | Polymer | Unrubbed | Rubbed | characteristics |
| 10′-1 | 18C₅O₉₅O | 0.6° | 88.1° | Excellent |
| 10′-3 | 18C₂₀O₈₀O | 87.1° | 89..8° | Excellent |
| 10′-4 | 18C₃₀O₇₀O | 87.6° | 89.9° | Excellent |
| 10′-5 | 18C₄₀O₆₀O | 87.7° | 89.1° | Excellent |
| 11′-1 | 12C₁₀O₉₀O | 5.9° | 88.0° | Excellent |
| 13′-3 | 18C₂₀O₈₀F | 88.2° | 88.1° | Excellent |
| 13′-4 | 18C₃₀O₇₀F | 88.9° | 89.8° | Excellent |
| 13′-5 | 18C₄₀O₆₀F | 88.3° | 89.6° | Excellent |
| 15′-1 | 18C₅O₉₅BT | 2.6° | 0.36° | Excellent |
| 15′-4 | 18C₃₀O₇₀BT | 88.8° | 87.7° | Excellent |
| 15′-5 | 18C₄₀O₆₀BT | 88.6° | 88.1° | Excellent |
| 16′-1 | 12C₅O₉₅BT | 1° | 85.2° | Excellent |
| 16′-2 | 12C₁₀O₉₀BT | 11.9° | 88.7° | Excellent |
| 16′-4 | 12C₃₀O₇₀BT | 88.7° | 85.4° | Excellent |
| 16′-5 | 12C₄₀O₆₀BT | 88.7° | 87.7° | Excellent |
| 17′-3 | 6C₂₀O₈₀BT | 6° | 9.9° | Excellent |
| 17′-4 | 6C₃₀O₇₀BT | 0.6° | 5.0° | Excellent |

TABLE 10
Reliability Testing for Pre-tilting angle

| Embodi- | | Before annealing | | After annealing^{a} | |
|---|---|---|---|---|---|
| ments | Polymer | Unrubbed | Rubbed | Unrubbed | Rubbed |
| 7′-1 | 18C₅O₉₅C | 88.6° | 89.7° | — | 87.1° |
| 7′-2 | 18C₁₀O₉₀C | 88.3° | 88.5° | — | 87.5° |
| 7′-3 | 18C₂₀O₈₀C | 88.5° | 88.8° | — | 87.8° |
| 7′-4 | 18C₃₀O₇₀C | 88.6° | 89.5° | — | 88.6° |
| 7′-5 | 18C₄₀O₆₀C | 88.5° | 89.6° | — | 88.7° |
| 1′-1 | 18C₅O₉₅B | 88.5° | 89.9° | — | 88.7° |
| 1′-2 | 18C₁₀O₉₀B | 88.1° | 89.6° | — | 87.3° |
| 1′-3 | 18C₂₀O₈₀B | 87.7° | 87.7° | — | 88.5° |
| 1′-4 | 18C₃₀O₇₀B | 88.0° | 88.1° | — | 88.5° |
| 1′-5 | 18C₄₀O₆₀B | 88.4° | 88.9° | — | 88.9° |
| 4′-1 | 18C₅O₉₅P | 87.3° | 89.0° | 89.0° | 87.2° |
| 4′-2 | 18C₁₀O₉₀P | 88.5° | 89.3° | 89.2° | 87.6° |
| 4′-3 | 18C₂₀O₈₀P | 88.5° | 89.5° | 89.1° | 88.4° |
| 4′-4 | 18C₃₀O₇₀P | 89.1° | 89.2° | 88.7° | 88.6° |
| 4′-5 | 18C₄₀O₆₀P | 88.9° | 89.6° | 89.8° | 89.1° |
| 10′-3 | 18C₂₀O₈₀O | 87.1° | 89..8° | — | 89.1° |
| 10′-4 | 18C₃₀O₇₀O | 87.6° | 89.9° | — | 89.4° |
| 10′-5 | 18C₄₀O₆₀O | 87.7° | 89.1° | — | 89.2° |
| 13′-3 | 18C₂₀O₈₀F | 88.2° | 88.1° | — | deteriorate^{b} |
| 13′-4 | 18C₃₀O₇₀F | 88.9° | 89.8° | — | deteriorate |
| 13′-5 | 18C₄₀O₆₀F | 88.3° | 89.6° | — | deteriorate |

^{a}Annealing at 60° C. for 72 hours
^{b}L.C alignment deteriorated after annealing

What is claimed is:

1. A chemical compound of formula (A):

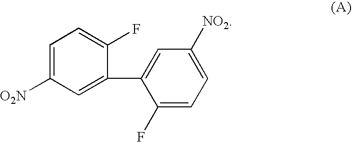

2. A diamine compound of formula (I) formed by reacting the compound of claim 1 with an alkyl alcohol (R₁OH) under an alkaline condition, and then reducing the dinitro-compound: 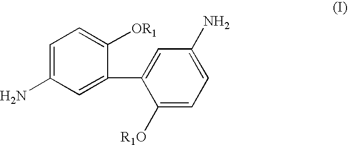
wherein R₁ is a C₂˜C₂₂ alkyl group.

3. A polyimide of formula (II) derived by reacting the diamine compound of claim 2 with a dianhydride containing Ar₁: 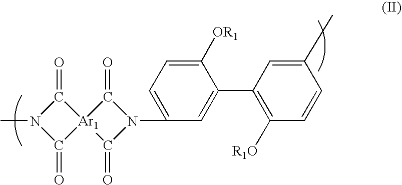
wherein Ar₁ is selected from a group consisting of

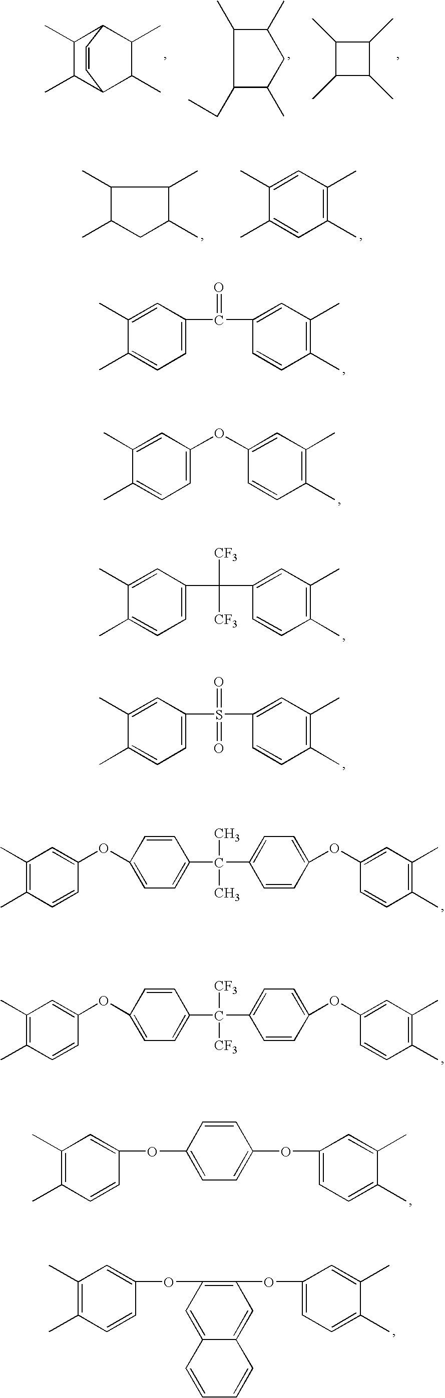

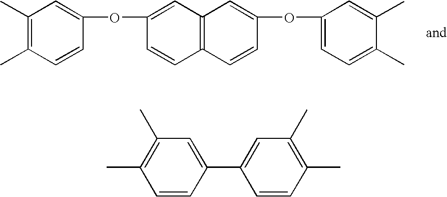

4. A polyimide as claimed in claim 3, wherein the polyimide is dissolvable in a solvent selected from a group consisting of NMP, DMAc, r-butyrolactone, m-cresol, o-chlorophenol, THF, and cyclohexanone.

5. A polyimide as claimed in claim 4, wherein the polyimide is dissolvable in NMP.

6. A polyimide as claimed in claim 5, wherein a viscosity of the polyimide is ranged from 0.05 to 3.0 dL/g.

7. A polyimide copolymer of formula (III) formed by reacting the diamine compound of claim 2 with a diamine compound containing Ar₂ and a dianhydride containing Ar₁: 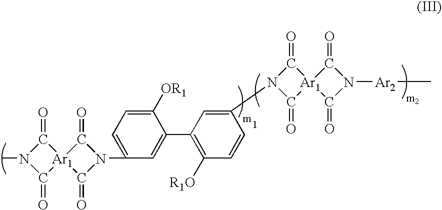
where in m₁ varise from 100 to 1 while m₂ varies correspondingly from 0 to 99; and Ar₁ is selected from a group consisting of 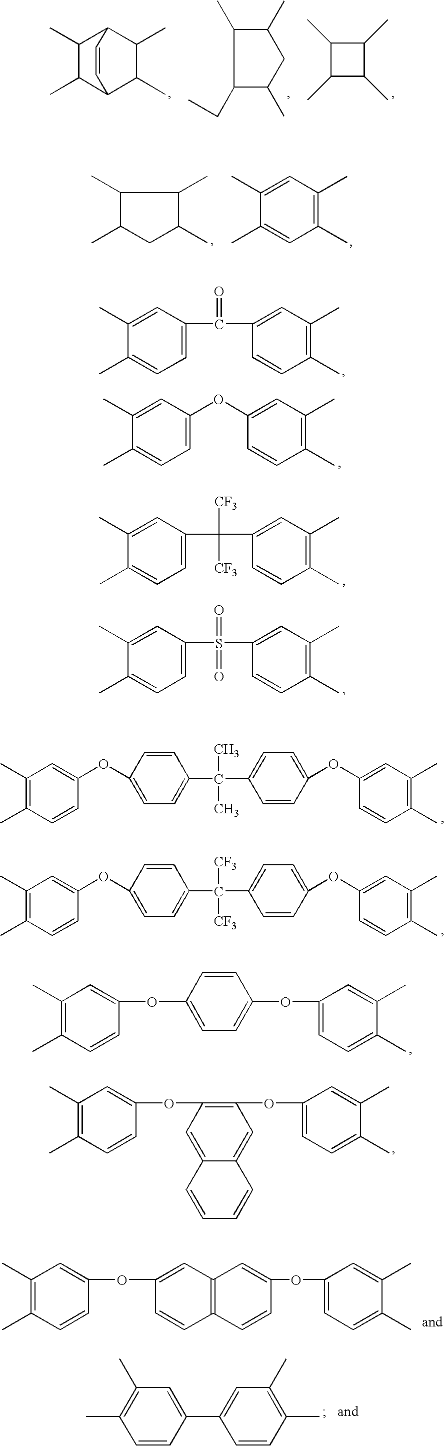
Ar₂ is selected from a group consisting of

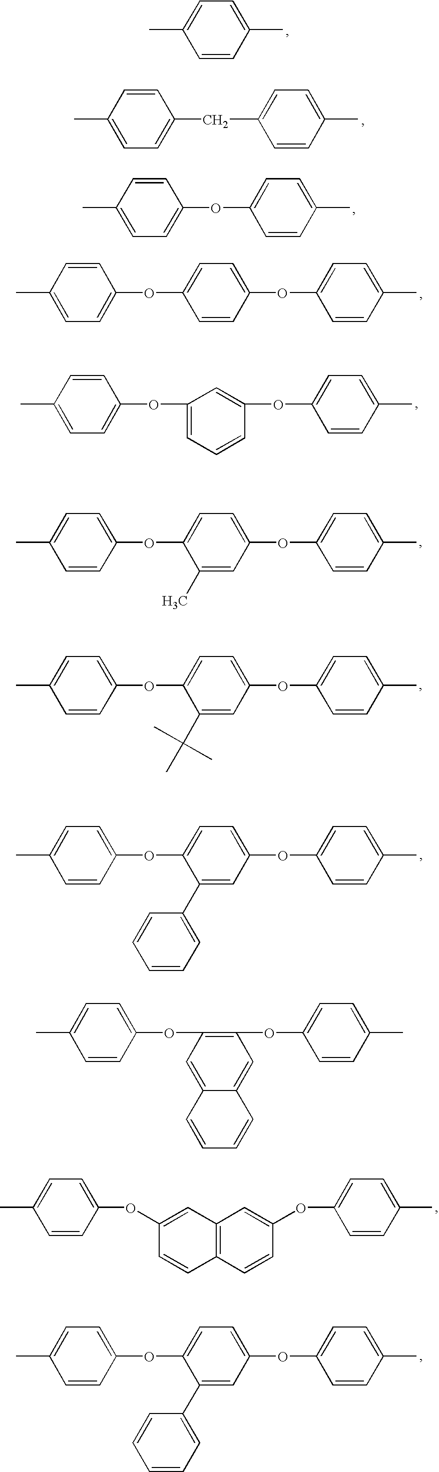

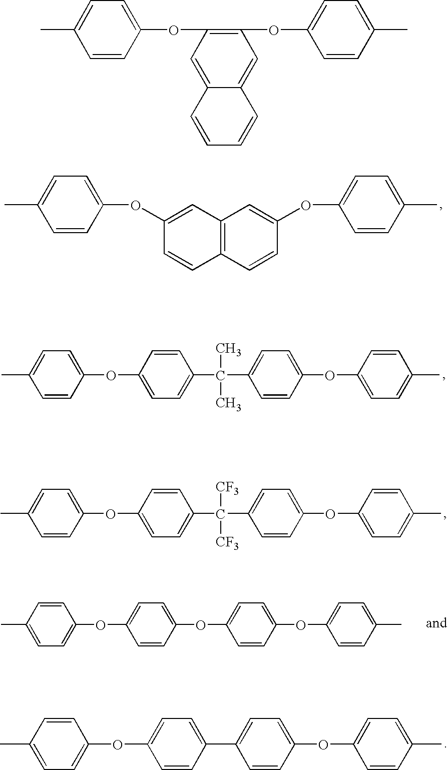

8. A polyimide as claimed in claim 7, wherein the polyimide copolymer is dissolvable in a solvent selected from a group consisting of NMP, DMAc, r-butyrolactone, m-cresol, o-chlorophenol, THF, and cyclohexanone.

9. A polyimide copolymer as claimed in claim 8, wherein the polyimide is dissolvable in NMP.

10. A polyimide copolymer as claimed in claim 9, wherein a viscosity of the polyimide is ranged from 0.05 to 3.0 dL/g.

11. A polyimide copolymer as claimed in claim 7, wherein the polyimide of formula(II) occupies 1-100% by weight.

12. A liquid alignment film comprising the polyimide of formula (II) of claim 2.

13. A liquid alignment film as claimed in claim 12, wherein the concentration of the polyimide of formula (II) is ranged from 1 to 10%.

14. A liquid alignment film as claimed in claim 13, wherein the concentration of the polyimide of formula (II) is lower than 7%.

15. A liquid alignment film comprising the polyimide copolymer of formula (III) of claim 7.

16. A liquid alignment film as claimed in claim 15, wherein the concentration of the polyimide copolymer of formula (III) is ranged from 1 to 10%.

17. A liquid alignment film as claimed in claim 16, wherein the concentration of the polyimide copolymer of formula (III) is lower than 7%.
